(19) United States
(12) Patent Application Publication
(10) Pub. No.: US 2020/0083597 A1
(43) Pub. Date: Mar. 12, 2020

(54) ANTENNA HOLDER AND ANTENNA UNIT

(71) Applicant: OLYMPUS CORPORATION, Tokyo (JP)

(72) Inventor: Yukako KATO, Tokyo (JP)

(73) Assignee: OLYMPUS CORPORATION, Tokyo (JP)

(21) Appl. No.: 16/683,865

(22) Filed: Nov. 14, 2019

Related U.S. Application Data
(63) Continuation of application No. PCT/JP2018/016203, filed on Apr. 19, 2018.

(30) Foreign Application Priority Data
Jun. 13, 2017 (JP) 2017-116179

(51) Int. Cl. H01Q 1/27; A61B 1/00; A61B 1/04

(52) U.S. Cl. CPC H01Q 1/273; A61B 1/00009; A61B 1/041; A61B 1/00016

(57) ABSTRACT

An antenna holder is configured to hold a receiving antenna including at least one receiver to receive a wireless signal transmitted from a medical device inserted into a subject. The antenna holder includes: a main body to which the receiving antenna is detachably attached; and a shield configured to reduce an electromagnetic wave. When the receiving antenna is attached to the main body, a part of the main body on a subject side, the receiving antenna, and the shield are serially arranged from the subject side.

### CROSS-REFERENCE TO RELATED APPLICATION

This application is a continuation of PCT International Application No. PCT/JP2018/016203 filed on Apr. 19, 2018, which designates the United States, incorporated herein by reference, and which claims the benefit of priority from Japanese Patent Application No. 2017-116179, filed on Jun. 13, 2017, incorporated herein by reference.

### BACKGROUND

### 1. Technical Field

The present disclosure relates to an antenna holder to hold a receiving antenna that receives a wireless signal, and an antenna unit.

### 2. Related Art

In the relater art, a capsule endoscope device having an imaging function, a wireless communication function, or the like in a capsule-shaped casing formed in a size with which insertion into a gastrointestinal tract of a subject such as a patient is possible has been known in a field of an endoscope. This capsule endoscope device generates image data by serially capturing an inside of the subject while moving in the inside of the subject such as an inside of a gastrointestinal tract by a peristalsis or the like after being swallowed from a mouth of the subject, and serially transmits this image data wirelessly.

The image data transmitted by the capsule endoscope device wirelessly in such a manner is received by a receiving device through a plurality of receiving antennas provided outside the subject. Each of the plurality of receiving antennas is held by an antenna holder and is fixed on a surface of a body of the subject (see, for example, Japanese Laid-open Patent Publication No. 2016-182215). In Japanese Laid-open Patent Publication No. 2016-182215, a subject wears an electromagnetic wave protective suit knitted with a fiber having a conductive property in a state in which a receiving antenna is fixed to a surface of a body, whereby deterioration in communication quality due to a noise from the outside is controlled.

### SUMMARY

In some embodiments, provided is an antenna holder configured to hold a receiving antenna including at least one receiver to receive a wireless signal transmitted from a medical device inserted into a subject. The antenna holder includes: a main body to which the receiving antenna is detachably attached; and a shield configured to reduce an electromagnetic wave. When the receiving antenna is attached to the main body, a part of the main body on a subject side, the receiving antenna, and the shield are serially arranged from the subject side.

In some embodiments, an antenna unit includes: a receiving antenna including at least one receiver to receive a wireless signal transmitted from a medical device inserted into a subject; and an antenna holder configured to hold the receiving antenna, the antenna holder being attached to the subject. The antenna holder includes a main body to which the receiving antenna is detachably attached, and a shield that reduces an electromagnetic wave, and when the receiving antenna is attached to the main body, a part of the main body on a subject side, the receiving antenna, and the shield are serially arranged from the subject side.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic view illustrating a schematic configuration of a capsule endoscope system including an antenna holder according to a first embodiment of the disclosure;

FIG. 2 is a schematic view illustrating a schematic configuration of the antenna holder illustrated in FIG. 1;

FIG. 3 is a schematic view illustrating a schematic configuration of a receiving antenna illustrated in FIG. 1;

FIG. 4 is an enlarged view of an antenna portion in FIG. 2;

FIG. 5 is an A-A line partial sectional view of FIG. 4;

FIG. 6 is a partial sectional view schematically illustrating a configuration of a main portion of an antenna holder according to a first modification example of the first embodiment of the disclosure;

FIG. 7 is a partial sectional view schematically illustrating a configuration of a main portion of an antenna holder according to a second modification example of the first embodiment of the disclosure;

FIG. 8 is a view schematically illustrating a configuration of a main portion of an antenna holder according to a third modification example of the first embodiment of the disclosure;

FIG. 9 is a schematic view illustrating a configuration of an antenna holder according to a second embodiment of the disclosure;

FIG. 10 is a B-B line partial sectional view of FIG. 9;

FIG. 11 is a partial sectional view schematically illustrating a configuration of a main portion of an antenna holder according to a first modification example of the second embodiment of the disclosure;

FIG. 12 is a partial sectional view schematically illustrating a configuration of a main portion of an antenna holder according to a second modification example of the second embodiment of the disclosure;

FIG. 13 is a view schematically illustrating a configuration of a main portion of an antenna holder according to a third modification example of the second embodiment of the disclosure;

FIG. 14 is a schematic view illustrating a schematic configuration of an antenna holder according to a third embodiment of the disclosure;

FIG. 15 is a partial sectional view schematically illustrating a configuration of the antenna holder according to the third embodiment of the disclosure;

FIG. 16 is a partial sectional view schematically illustrating a configuration of a main portion of an antenna holder according to a first modification example of the third embodiment of the disclosure;

FIG. 17 is a partial sectional view schematically illustrating a configuration of a main portion of an antenna holder according to a second modification example of the third embodiment of the disclosure;

FIG. 18 is a schematic view illustrating a schematic configuration of an antenna holder according to a fourth embodiment of the disclosure;

FIG. 19 is a C-C line partial sectional view of FIG. 18;

FIG. 20 is a partial sectional view schematically illustrating a configuration of a main portion of an antenna holder according to a first modification example of the fourth embodiment of the disclosure; and

FIG. 21 is a partial sectional view schematically illustrating a configuration of a main portion of an antenna holder according to a second modification example of the fourth embodiment of the disclosure.

### DETAILED DESCRIPTION

In the following, a capsule endoscope system using a capsule endoscope that is a medical device will be described as an embodiment according to the disclosure. Note that the same sign is assigned to identical parts in description of the drawings. Also, it is necessary to note that the drawings are schematic and a relationship between a thickness and a width of each member, a proportion of each member, and the like are different from the reality.

### First Embodiment

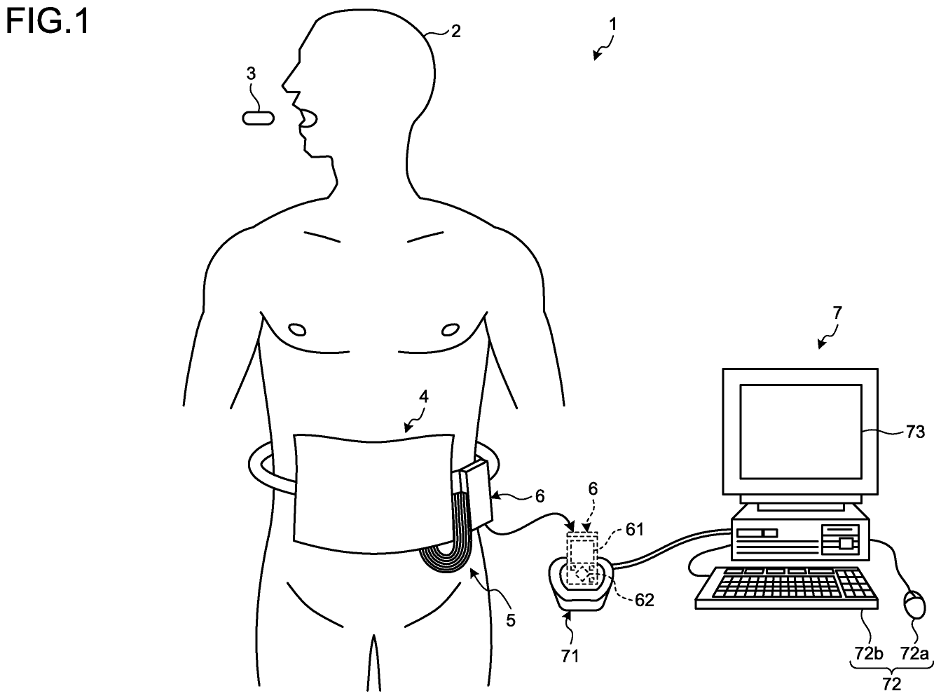

FIG. 1 is a schematic view illustrating a schematic configuration of a capsule endoscope system including an antenna holder according to the first embodiment of the disclosure. A capsule endoscope system 1 illustrated in FIG. 1 includes a capsule endoscope device 3 as a medical device inserted into a subject 2, an antenna holder 4 that is attached to the subject 2 and that holds a plurality of receiving antennas, an antenna device 5 that is attached to the antenna holder 4 and that includes a plurality of receiving antennas to receive a wireless signal transmitted from the capsule endoscope device 3 inserted into the subject 2, a receiving device 6 to which the antenna device 5 is detachably connected and which performs predetermined processing with respect to the wireless signal received by the antenna device 5 and records or displays the signal, and an image processing device 7 that performs processing corresponding to image data of an inside of the subject 2 captured by the capsule endoscope device 3 and/or displays the image data. An antenna unit includes the antenna holder 4 and the receiving antennas included in the antenna device 5.

The capsule endoscope device 3 has an imaging function to capture an inside of the subject 2, and a wireless function to transmit a wireless signal including image data acquired by capturing the inside of the subject 2 to a receiving antenna 51. The capsule endoscope device 3 passes through an esophagus in the inside of the subject 2 by being swallowed into the subject 2, and moves inside the subject 2 by a peristalsis of a lumen of a gastrointestinal tract. The capsule endoscope device 3 successively captures the inside of the subject 2 in a minute time interval such as an interval of 0.5 seconds (such as 2 fps) while moving inside the subject 2, generates image data of the captured inside of the subject 2, and serially transmits the image data to the antenna device 5. For example, the capsule endoscope device 3 outputs a wireless signal at a frequency equal to or higher than 300 MHz and equal to or lower than 450 MHz.

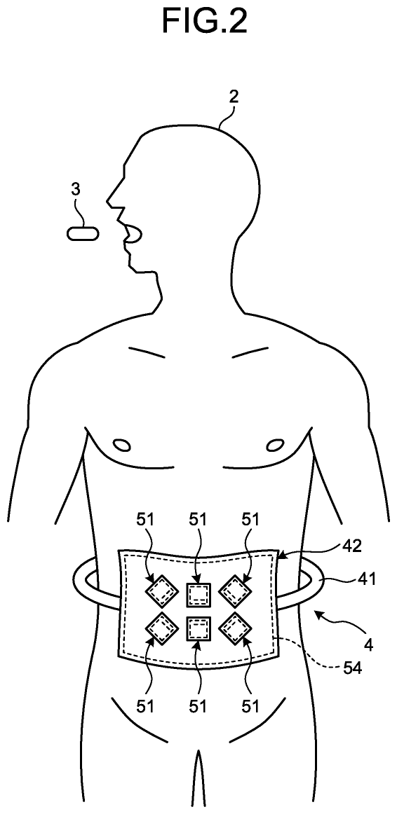

FIG. 2 is a schematic view illustrating a schematic configuration of the antenna holder illustrated in FIG. 1, and is a view illustrating a state in which the antenna holder 4 is attached to the subject 2. As illustrated in FIG. 2, the antenna holder 4 includes a belt portion 41 that fixes the antenna holder 4 to the subject 2, and an antenna attachment portion 42 which is supported by the belt portion 41 and to which the plurality of receiving antennas 51 is attached.

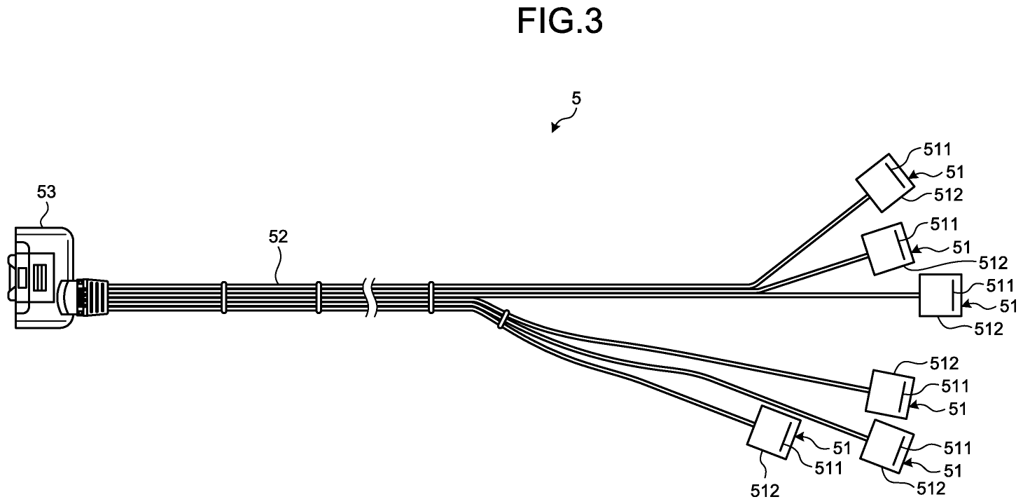

Here, a configuration of the antenna device 5 will be described with reference to FIG. 3. FIG. 3 is a schematic view illustrating a schematic configuration of a receiving device illustrated in FIG. 1. As illustrated in FIG. 3, the antenna device 5 includes the plurality of receiving antennas 51 that receives a wireless signal from the capsule endoscope device 3, an antenna cable 52 that propagates the wireless signal received by the plurality of receiving antennas 51 to the receiving device 6, and a connector portion 53 connected to the receiving device 6.

Each of the receiving antennas 51 includes an antenna pattern 511 as a receiver to receive the wireless signal from the capsule endoscope device 3, and a base 512 in which a wiring pattern is formed. The wiring pattern formed in the base 512 outputs the wireless signal received by the antenna pattern 511 to the receiving device 6. The base 512 may include a bendable flexible substrate, or may include a rigid substrate having rigidity.

Referring back to FIG. 2, the belt portion 41 of the antenna holder 4 fixes the antenna holder 4 along a surface of a body of the subject 2 by being attached to the subject 2. The belt portion 41 is preferably formed of an elastic material such as rubber or a polyurethane elastic fiber in such a manner that the antenna holder 4 can be fixed according to a body type of a subject.

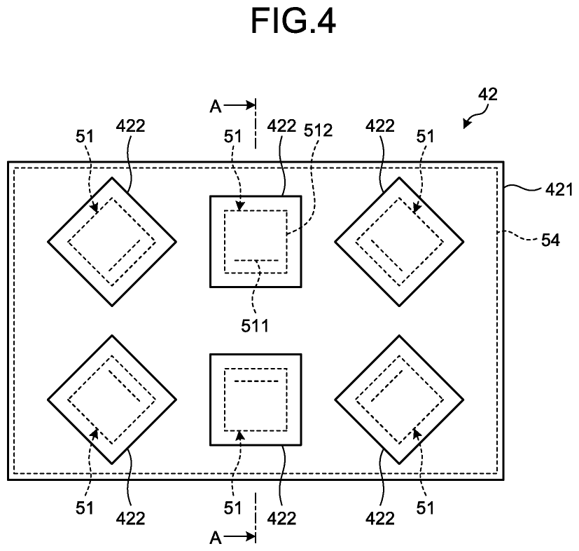

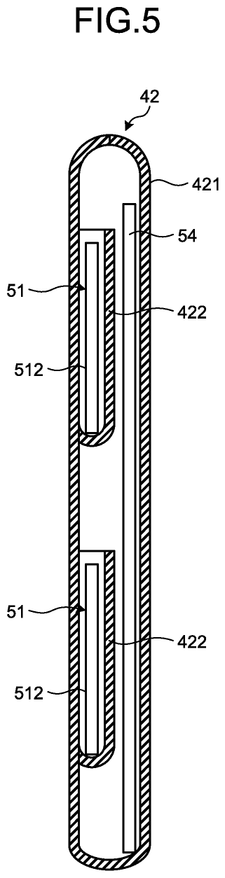

FIG. 4 is an enlarged view of the antenna attachment portion 42 in FIG. 2. FIG. 5 is an A-A line partial sectional view of the antenna attachment portion 42 illustrated in FIG. 4. In FIG. 5, the subject 2 is arranged on a left side of the antenna attachment portion 42. In the following, description will be made on the assumption that the subject 2 is also arranged on the left side in a different partial sectional view. As illustrated in FIGS. 4 and 5, the antenna attachment portion 42 includes a main body 421 that houses the plurality of receiving antennas 51 and a shield 54, and six antenna fixing portions 422 which are provided in the main body 421 and each of which holds one receiving antenna 51 and fixes a position thereof. In the following, description will be made on the assumption that the number of antenna fixing portions 422 is six and six receiving antennas 51 are attached. However, the number of antenna fixing portions 422 and that of the receiving antennas 51 are not limited to six. Also, the number of the antenna fixing portions 422 and that of the receiving antennas 51 are not necessarily identical.

The main body 421 has a saclike shape formed of cloth or the like. The main body 421 has an openable/closable opening formed by utilization of a snap fastener, a slide fastener, or a hook and loop fastener, for example.

Each antenna fixing portion 422 includes cloth formed into a pocked shape, or the like and houses one receiving antenna 51 inside. Each antenna fixing portion 422 is correspondingly provided in a position where the receiving antenna 51 is arranged.

The shield 54 reduces passing of an electromagnetic wave. In the present description, “reduction of an electromagnetic wave” includes complete shielding to completely prevent passing of an electromagnetic wave. For example, the shield 54 is produced by knitting of a conductive fiber in a net-like manner. The shield 54 may be formed of an electromagnetic wave shielding material instead of being formed of a conductive fiber. The shield 54 may be a conductive metal plate, sheet, or non-woven cloth. Note that the shield 54 may be what reduces an electromagnetic wave in a specific frequency band.

At least one of corrosion preventing processing or water-resistance processing may be performed on the shield 54. In the corrosion preventing processing, processing to prevent rust is performed, for example, by surface treatment. In the water-resistance processing, processing of preventing a conductive fiber or the like from touching water is performed by surface treatment such as coating. Since corrosion preventing processing or water-resistance processing is performed on the shield 54, it is possible to wash the main body 421 in water even when the shield 54 is kept housed in the main body 421. Here, the receiving antennas 51 are removed.

The shield 54 has a size with which it is possible to cover faces on one side of the bases 512 of all receiving antennas 51 when being housed in the main body 421. The shield 54 has a size larger than that of a face with which the shield 54 covers the bases 512. Note that the shield 54 only needs to have a size equal to or larger than the face that covers the base 512. In the following, a “size with which covering is possible” means a size equal to or larger than a size of a covered face. In a case where the bases 512 are a flexible substrate, the shield 54 has a size, when a face of the shield 54 is projected on the bases 512, including the bases 512 in this projection region. When the antenna holder 4 is attached to the subject 2, the shield 54 is arranged on a side opposite to the subject 2 with respect to the receiving antennas 51. That is, the shield 54 covers a face on an opposite side of a subject 2 side of each receiving antenna 51. In a state in which the antenna holder 4 is attached to the subject 2, a part of the main body 421 which part is on a side of being attached to the subject 2, the receiving antennas 51, the shield 54, and a part of the main body 421 which part is on an opposite side of the side attached to the subject 2 are serially arranged from the subject 2 side.

The receiving device 6 records image data of the inside of the subject 2 which data is included in a wireless signal transmitted from the capsule endoscope device 3 through the plurality of receiving antennas 51 or displays an image corresponding to the image data of the inside of the subject 2. The receiving device 6 includes a reception display unit 61 that displays an image corresponding to the image data, and an operating unit 62 that receives an input of an instruction signal to operate the receiving device 6 and information related to a position of each receiving antenna 51. Also, the receiving device 6 receives, through each receiving antenna 51, a wireless signal transmitted from the capsule endoscope device 3, calculates and records received strength (received electric field strength) of this received wireless signal for each receiving antenna 51, and estimates a position of the capsule endoscope device 3 in the subject 2. The receiving device 6 records image data included in the wireless signal received from the capsule endoscope device 3, received strength of the wireless signal received by each receiving antenna 51, and time information of the image data generated by the capsule endoscope device 3 in association.

The image processing device 7 displays an image corresponding to the image data of the inside of the subject 2 which data is acquired through the receiving device 6. The image processing device 7 includes a cradle 71 to read image data or the like from the receiving device 6, an operation input unit 72 such as a mouse 72a or a keyboard 72b, and a display unit 73 to display an image corresponding to the image data. When the receiving device 6 is attached, the cradle 71 acquires, from the receiving device 6, image data, received strength of each receiving antenna 51 which strength is associated with this image data, time information of the image data generated by the capsule endoscope device 3, identification information of the capsule endoscope device 3, and the like and transfers the acquired various kinds of information to the image processing device 7. The operation input unit 72 receives an input by a user. While operating the operation input unit 72 and looking at an image of the inside of the subject 2 which image is serially displayed by the image processing device 7, the user observes a body part inside the subject 2 such as an esophagus, a stomach, a small intestine, and a large intestine, and diagnoses the subject 2.

In the antenna holder 4 in the above-described first embodiment, the antenna fixing portions 422 respectively hold the plurality of receiving antennas 51, and the shield 54 is held in such a manner as to cover faces on one side of the bases 512 of all receiving antennas 51. According to the present first embodiment, faces on a side opposite to the subject 2 side of the receiving antennas 51 are shielded with the shield 54 and a noise from the outside is reduced. Thus, it is possible to control deterioration in communication quality while controlling a necessary amount of a material compared to an amount of fiber necessary for a protective suit. Note that even in a case where the shield 54 is produced by utilization of a material different from a fiber such as a metal plate, an amount of the material can be controlled compared to an amount of material for a protective suit since a shielded region is smaller than a shielded region by the protective suit.

Also, according to the above-described present first embodiment, it is not necessary to wear a protective suit in a conventional manner, and it is possible to reduce discomfort of a subject due to sticking of a protective suit, or the like. Also, since it is possible to produce the shield 54 with a small amount of fiber compared to an amount of fiber necessary for production of a protective suit, it is possible to control a production and distribution cost compared to a case where a protective suit is used.

### First Modification Example of First Embodiment

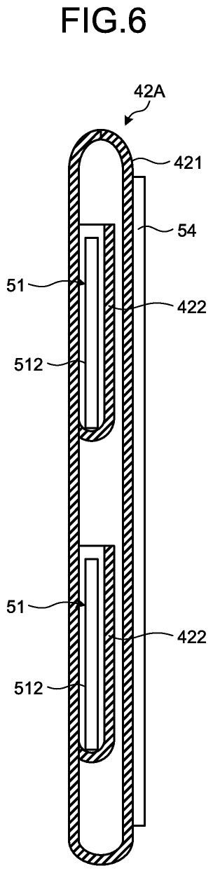

FIG. 6 is a partial sectional view schematically illustrating a configuration of a main portion of an antenna holder according to the first modification example of the first embodiment of the disclosure. The partial sectional view illustrated in FIG. 6 corresponds to the A-A line cross section illustrated in FIG. 4. In the above-described first embodiment, it has been described that a shield 54 is housed in a main body 421. However, a shield 54 is provided outside a main body 421 in the present first modification example. An antenna holder according to the present first modification example includes the above-described belt portion 41 and an antenna attachment portion 42A. The antenna attachment portion 42A includes the above-described main body 421, and six antenna fixing portions 422 which are provided in the main body 421 and each of which holds one receiving antenna 51 and fixes a position thereof. The shield 54 is attached to an outer surface of the main body 421 which outer surface is on an opposite side of a subject 2 side when the antenna holder is attached to a subject 2. The shield 54 is attached to the main body 421 by a publicly known fixing means such as an adhesive or a button.

In the present first modification example, in a state in which the antenna holder is attached to the subject 2, a part of the main body 421 which part is on a side of being attached to the subject 2, the receiving antennas 51, a part of the main body 421 which part is on an opposite side of the side attached to the subject 2, and the shield 54 are serially arranged from the subject 2 side.

The plurality of antenna fixing portions 422 holds the plurality of receiving antennas 51, and the shield 54 is provided in such a manner as to cover faces on one side of bases 512 of all of the receiving antennas 51 also in the present first modification example. Thus, faces on the subject 2 side and an opposite side thereof of the receiving antennas 51 are shielded with the shield 54, whereby it is possible to control deterioration in a communication quality with a simple configuration while controlling a necessary amount of a material.

### Second Modification Example of First Embodiment

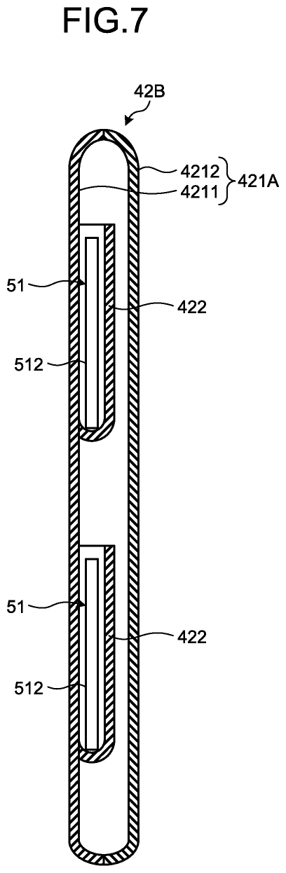

FIG. 7 is a partial sectional view schematically illustrating a configuration of a main portion of an antenna holder according to the second modification example of the first embodiment of the disclosure. The partial sectional view illustrated in FIG. 7 corresponds to the A-A line cross section illustrated in FIG. 4. In the above-described first embodiment, it has been described that a shield 54 is separated from a main body 421. However, a shield is included in a part of a main body in the present second modification example. The antenna holder according to the present second modification example includes the above-described belt portion 41, and an antenna attachment portion 42B. The antenna attachment portion 42B includes a main body 421A that houses a plurality of receiving antennas 51, and six antenna fixing portions 422 which are provided in the main body 421 and each of which holds one receiving antenna 51 and fixes a position thereof.

The main body 421A has a saclike shape. The main body 421A includes a partial main body 4211 which is included in a part of the saclike shape and in which the antenna fixing portions 422 are provided, and a shielding portion 4212 that reduces passing of an electromagnetic wave and that forms the saclike shape with the partial main body 4211. In the main body 421A, the partial main body 4211 is arranged on a subject 2 side when the antenna holder is attached to a subject 2. For example, the shielding portion 4212 is formed of a conductive fiber in a manner of being equivalent to the above-described shield 54. In the present second modification example, the shielding portion 4212 is placed on a face on an opposite side of the subject 2 side of the receiving antennas 51, and covers faces on the opposite side of the subject 2 side of bases 512 of all of the receiving antennas 51. The shielding portion 4212 corresponds to a shield.

In the present second modification example, in a state in which the antenna holder is attached to the subject 2, the partial main body 4211 that is a part of the main body 421A which part is on a side of being attached to the subject 2, the receiving antennas 51, and the shielding portion 4212 that is in common with a part of the main body 421A which part is on the opposite side of the side being attached to the subject 2 are serially arranged from the subject 2 side.

The plurality of antenna fixing portions 422 holds the plurality of receiving antennas 51, and the shielding portion 4212 is provided in such a manner as to cover faces on one side of bases 512 of all of the receiving antennas 51 also in the present second modification example. Thus, faces on the opposite side of the subject 2 side of the receiving antennas 51 are shielded with the shielding portion 4212, and it is possible to control deterioration in communication quality with a simple configuration while controlling a necessary amount of a material.

### Third Modification Example of First Embodiment

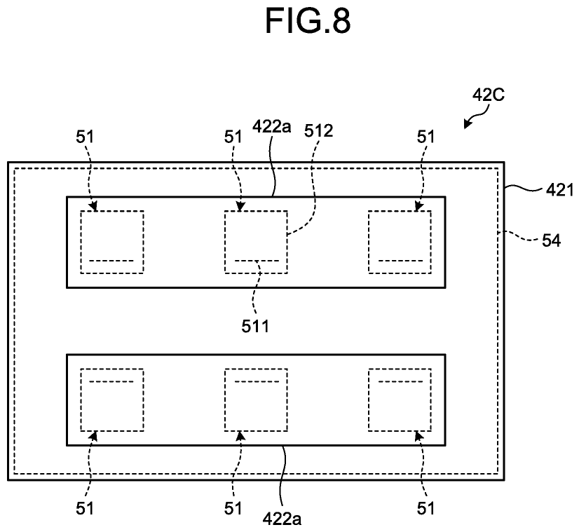

FIG. 8 is a view schematically illustrating a configuration of a main portion of an antenna holder according to the third modification example of the first embodiment of the disclosure. In the above-described first embodiment, it has been described that each antenna fixing portion 422 holds one receiving antenna 51. However, in an antenna attachment portion 42C according to the present third modification example, two antenna fixing portions 422a are provided and each antenna fixing portion 422a houses a plurality of (three in FIG. 8) receiving antennas 51. Even with such a configuration, it is possible to acquire the above-described effect by an arrangement of a shield 54. In the present third modification example, the antenna fixing portions 422a house the receiving antennas 51 different from each other.

### Second Embodiment

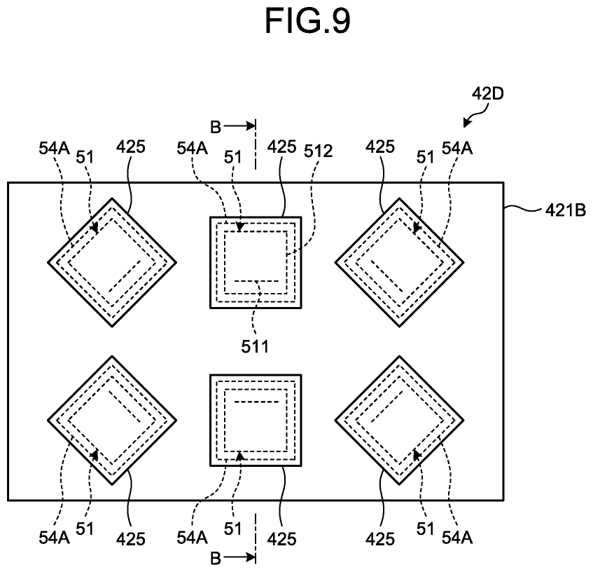

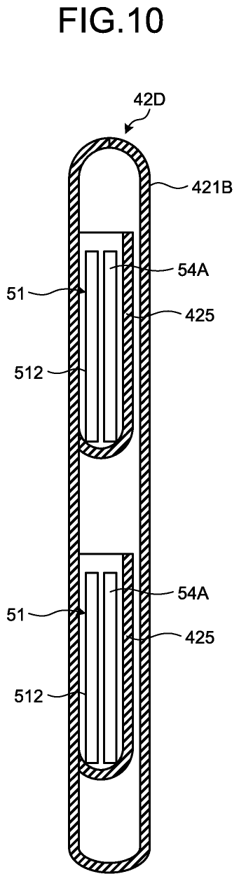

Next, the second embodiment of the disclosure will be described. FIG. 9 is a schematic view illustrating a configuration of an antenna holder according to the second embodiment of the disclosure. FIG. 10 is a B-B line partial sectional view of an antenna attachment portion illustrated in FIG. 9. In the above-described first embodiment, it has been described that one shield 54 covers a plurality of receiving antennas 51 together. However, a plurality of shields 54A individually covers a receiving antenna 51 in the present second embodiment.

An antenna holder according to the present second embodiment includes the above-described belt portion 41, and an antenna attachment portion 42D. As illustrated in FIGS. 9 and 10, the antenna attachment portion 42D includes a main body 421B that houses a plurality of receiving antennas 51 and a plurality of shields 54A, and six antenna fixing portions 425 which are provided in the main body 421B and each of which holds one receiving antenna 51 and shield 54A and fixes positions thereof.

The main body 421B has a saclike shape formed of cloth or the like. The main body 421B has an openable/closable opening formed by utilization of a snap fastener, a slide fastener, or a hook and loop fastener, for example.

Each antenna fixing portion 425 is cloth formed into a pocket shape, or the like and houses one each of the receiving antennas 51 and the shields 54A inside. Each antenna fixing portion 425 is provided according to an arrangement of the receiving antennas 51.

Each shield 54A has a mesh shape formed of a conductive fiber. The shield 54A is formed of the same material with the above-described shield 54. The shield 54A has a size with which it is possible to cover a base 512 of each receiving antenna 51.

The shield 54A has a size with which it is possible to cover, when being housed in an antenna fixing portion 425, a face on one side of a base 512 of a receiving antenna 51 housed in the same antenna fixing portion 425. When an antenna holder 4 is attached to a subject 2, a shield 54A is arranged on a side opposite to a subject 2 side with respect to a receiving antenna 51. That is, the shields 54A respectively cover faces on an opposite side of the subject 2 side of the receiving antennas 51.

In the above-described second embodiment, the plurality of antenna fixing portions 425 respectively houses the plurality of receiving antennas 51 and the plurality of shields 54A, and the shields 54A respectively cover faces on one side of the bases 512 of the receiving antennas 51. According to the present second embodiment, the faces on the opposite side of the subject 2 side of the receiving antennas 51 are respectively shielded with the shields 54A and a noise from the outside is reduced. Thus, it is possible to control deterioration in communication quality with a simple configuration while controlling a necessary amount of a material.

Also, similarly to the above-described first embodiment, according to the above-described present second embodiment, it is possible to reduce discomfort of a subject due to sticking of a protective suit, or the like and to control a production and distribution cost compared to a case where a protective suit is used.

Note that in the above-described second embodiment, a shield 54A may be attached to a surface on an outer side of a pocket formed by an antenna fixing portion 425.

### First Modification Example of Second Embodiment

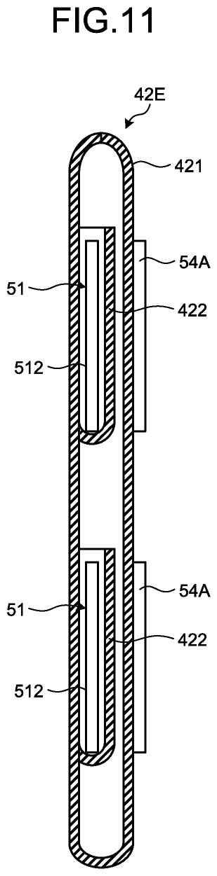

FIG. 11 is a partial sectional view schematically illustrating a configuration of a main portion of an antenna holder according to the first modification example of the second embodiment of the disclosure. The partial sectional view illustrated in FIG. 11 corresponds to the B-B line cross section illustrated in FIG. 9. In the above-described second embodiment, it has been described that each shield 54A is housed in a main body 421B. However, each shield 54A is provided outside of a main body 421B in the present first modification example. The antenna holder according to the present first modification example includes the above-described belt portion 41 and an antenna attachment portion 42E. The antenna attachment portion 42E includes the above-described main body 421, and six antenna fixing portions 422 which are provided in the main body 421 and each of which holds one receiving antenna 51 and fixes a position thereof. According to an arrangement of the receiving antennas 51, each shield 54A is attached to an outer surface of the main body 421 which outer surface is on an opposite side of a subject side when the antenna holder is attached to a subject 2. Each shield 54A is attached to the main body 421 by a publicly known fixing means such as an adhesive or a button.

The plurality of antenna fixing portions 422 holds the plurality of receiving antennas 51, and the plurality of shields 54A is provided in such a manner as to respectively cover faces on an opposite side of the subject 2 side of the receiving antennas 51 (base 512) also in the present first modification example. Thus, the faces on the opposite side of the subject 2 side of the receiving antennas 51 are shielded with the shields 54A, and it is possible to control deterioration in communication quality with a simple configuration while controlling a necessary amount of a material.

### Second Modification Example of Second Embodiment

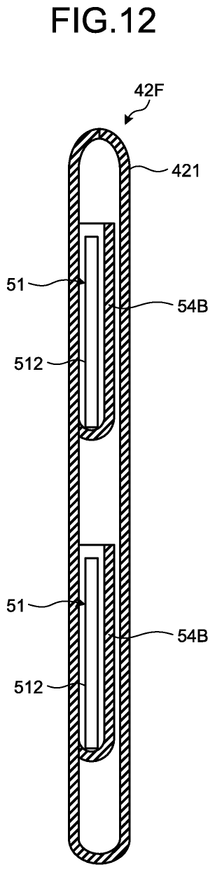

FIG. 12 is a partial sectional view schematically illustrating a configuration of a main portion of an antenna holder according to the second modification example of the second embodiment of the disclosure. The partial sectional view illustrated in FIG. 12 corresponds to the B-B line cross section illustrated in FIG. 9. In the above-described second embodiment, it has been described that a shield 54A is separated from an antenna fixing portion 425. However, a shield is provided as an antenna fixing portion in the present second modification example. The antenna holder according to the present second modification example includes the above-described belt portion 41, and an antenna attachment portion 42F. The antenna attachment portion 42F includes the above-described main body 421, and six shields 54B which are provided in the main body 421 and each of which holds one receiving antenna 51 and holds and fixes a position thereof.

Each shield 54B is formed in a pocket shape by utilization of a conductive fiber, and houses one receiving antenna 51 inside. The shield 54B has a function of an antenna fixing portion to fix a receiving antenna 51, and a shielding function to shield a face on an opposite side of a subject 2 side of the receiving antenna 51. Corrosion preventing processing or water-resistance processing is preferably performed on the shield 54B. Note that a part corresponding to a lower face of a pocket may be formed of cloth, and only a part facing a face on an opposite side of a subject 2 side of a receiving antenna 51 may be formed of a conductive fiber or the like.

The plurality of shields 54B respectively holds the plurality of receiving antennas 51 detachably, whereby a face on the opposite side of the subject 2 side of each receiving antenna 51 (base 512) is shielded also in the present second modification example. Thus, it is possible to control deterioration in communication quality with a simple configuration while controlling a necessary amount of a material.

### Third Modification Example of Second Embodiment

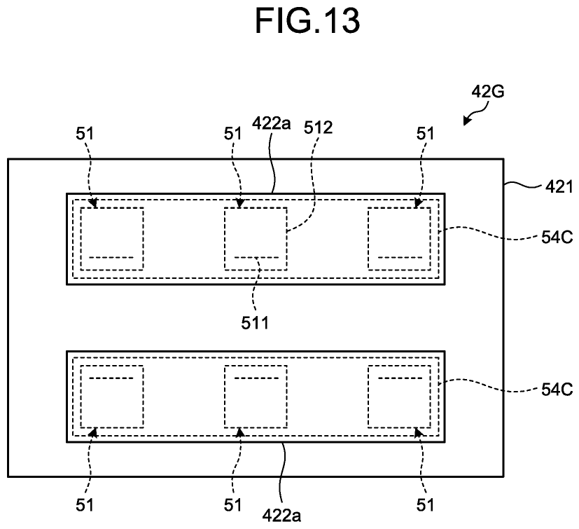

FIG. 13 is a view schematically illustrating a configuration of a main portion of an antenna holder according to the third modification example of the second embodiment of the disclosure. In the above-described second embodiment, it has been described that each antenna fixing portion 425 holds one receiving antenna 51. However, in an antenna attachment portion 42G according to the present third modification example, two antenna fixing portions 422a are provided, and each antenna fixing portion 422a houses a plurality of (three in FIG. 13) receiving antennas 51. In the present third modification example, two shields 54C respectively provided in the antenna fixing portions 422a are included. The shields 54C cover the receiving antennas 51 different from each other. The shield 54C according to the present third modification example has a size with which it is possible to cover bases 512 of three receiving antennas 51 held in the antenna fixing portion 422a. Even with a configuration in a manner of the present third modification example, it is possible to acquire the above-described effect by an arrangement of a shield 54.

### Third Embodiment

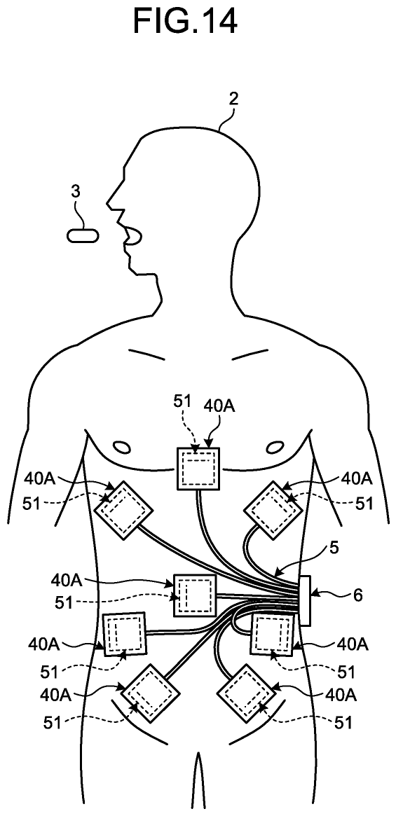

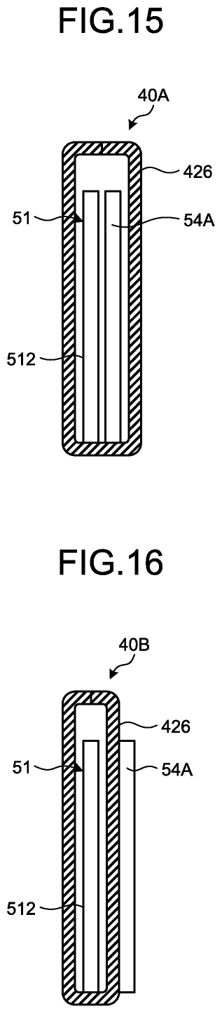

Next, the third embodiment of the disclosure will be described. FIG. 14 is a schematic view illustrating a configuration of an antenna holder according to the third embodiment of the disclosure and is a view illustrating a state in which the antenna holder is attached to a subject 2. FIG. 15 is a partial sectional view of the antenna holder and is a partial sectional view in which a plane orthogonal to a face attached to the subject 2 (such as face facing subject 2 of base 512) is a cross section. A capsule endoscope system according to the present third embodiment includes a capsule endoscope device 3, a plurality of antenna holders 40A that is attached to a subject 2 and that respectively holds a plurality of receiving antennas 51, an antenna device 5 that is attached to each of the antenna holders 40A and that includes the plurality of receiving antennas 51 to receive a wireless signal transmitted from the capsule endoscope device 3 inserted into the subject 2, a receiving device 6, and an image processing device 7 (see FIG. 1).

As illustrated in FIG. 15, each antenna holders 40A includes a main body 426 in a saclike shape formed of cloth or the like. The main body 426 houses one each of the receiving antennas 51 and shields 54A. The plurality of antenna holders 40A is independent from each other and is respectively pasted to predetermined positions of the subject 2.

Each shield 54A has a size with which it is possible to cover, when being housed in an antenna holder 40A, a face on one side of a receiving antenna 51 housed in the same antenna holder 40A. When the antenna holders 40A are attached to the subject 2, the shields 54A are arranged on a side opposite to the subject 2 with respect to the receiving antennas 51. That is, the shields 54A respectively cover faces on an opposite side of the subject 2 side of the receiving antennas 51.

In the above-described third embodiment, the plurality of antenna holders 40A respectively house the plurality of receiving antennas 51 and the plurality of shields 54A, and the shields 54A respectively cover faces on one side of bases 512 of the receiving antennas 51. According to the present third embodiment, the faces on the opposite side of the subject 2 side of the receiving antennas 51 are shielded with the shields 54A and a noise from the outside is reduced. Thus, it is possible to control deterioration in communication quality while controlling a necessary amount of a material.

Also, similarly to the above-described first embodiment, according to the above-described present third embodiment, it is possible to reduce discomfort of a subject due to sticking of a protective suit, or the like and to control a production and distribution cost compared to a case where a protective suit is used.

### First Modification Example of Third Embodiment

FIG. 16 is a partial sectional view schematically illustrating a configuration of a main portion of an antenna holder according to the first modification example of the third embodiment of the disclosure. In the above-described third embodiment, it has been described that each shield 54A is housed in an antenna holder 40A. However, each shield 54A is provided outside an antenna holder 40B in the present first modification example. The antenna holder 40B according to the present first modification example houses a receiving antenna 51. To an outer surface that is on an opposite side of a subject side when the antenna holder is attached to a subject 2, a shield 54A is attached. Each shield 54A is attached to a main body 426 by a publicly known fixing means such as an adhesive or a button.

A plurality of antenna holders 40B respectively holds a plurality of receiving antennas 51, and a plurality of shields 54A is provided on outer surfaces of the antenna holders 40B in such a manner as to cover faces on an opposite side of a subject side of the receiving antennas 51 also in the present first modification example. Thus, the faces on the opposite side of the subject 2 side of the receiving antennas 51 (base 512) are shielded with the shields 54A, and it is possible to control deterioration in communication quality while controlling a necessary amount of a material.

### Second Modification Example of Third Embodiment

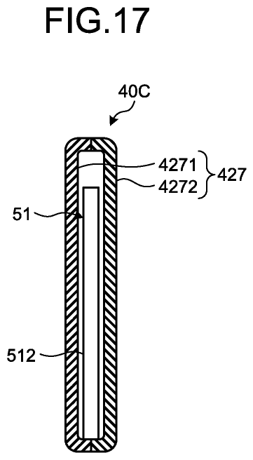

FIG. 17 is a partial sectional view schematically illustrating a configuration of a main portion of an antenna holder according to the second modification example of the third embodiment of the disclosure. In the above-described third embodiment, it has been described that a shield 54A is separated from an antenna holder 40A. However, a shield is provided as a main body in the present second modification example. An antenna holder 40C according to the present second modification example includes a main body 427 to house a receiving antenna 51.

The main body 427 has a saclike shape. The main body 427 is formed of cloth or the like, and includes a partial main body 4271 included in a part of the saclike shape, and a shielding portion 4272 that reduces an electromagnetic wave and that forms the saclike shape with the partial main body 4271. In the main body 427, the partial main body 4271 is arranged on a subject 2 side when the antenna holder 40C is attached to a subject 2. For example, the shielding portion 4272 is formed of a conductive fiber in a manner of being equivalent to the above-described shield 54. In the present second modification example, the shielding portion 4272 is placed on a face on an opposite side of the subject 2 side of the receiving antenna 51 and covers faces on the opposite side of the subject 2 side of all receiving antennas 51 (base 512). The shielding portion 4272 corresponds to a shield.

A plurality of antenna holders 40C respectively holds a plurality of receiving antennas 51, and a plurality of shielding portions 4272 is provided in such a manner as to respectively cover faces on an opposite side of a subject side of the receiving antennas 51 also in the present second modification example. Thus, the faces on the opposite side of the subject 2 side of the receiving antennas 51 are shielded with the shielding portions 4272, and it is possible to control deterioration in communication quality while controlling a necessary amount of a material.

### Fourth Embodiment

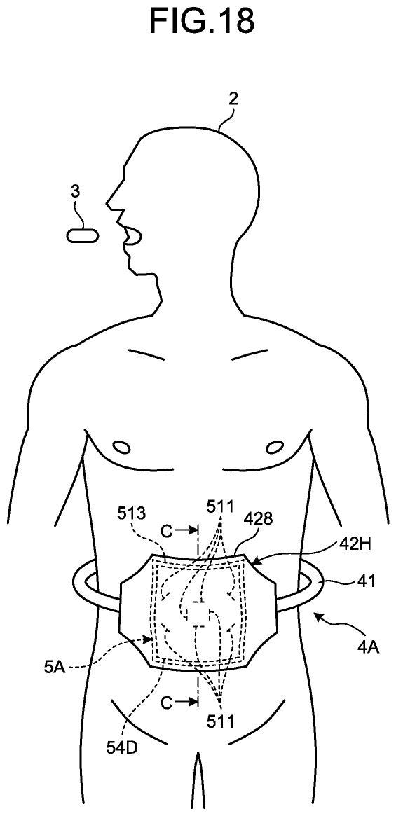

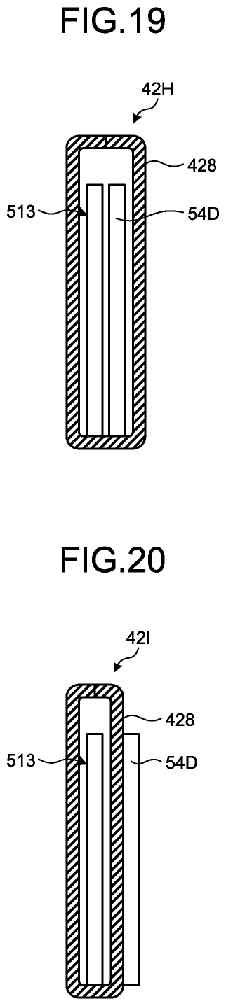

Next, the fourth embodiment of the disclosure will be described. FIG. 18 is a schematic view illustrating a configuration of an antenna holder according to the fourth embodiment of the disclosure, and is a view illustrating a state in which the antenna holder is attached to a subject 2. FIG. 19 is a C-C line partial sectional view of an antenna attachment portion illustrated in FIG. 18. A capsule endoscope system according to the present fourth embodiment includes a capsule endoscope device 3, an antenna holder 4A that is attached to the subject 2 and that holds a base 513 in which a plurality of antenna pattern 511 is formed, an antenna device 5A that is attached to the antenna holder 4A and that includes the base 513, a receiving device 6, and an image processing device 7 (see FIG. 1).

The antenna holder 4A according to the present fourth embodiment includes the above-described belt portion 41, and an antenna attachment portion 42H. The antenna attachment portion 42H includes the base 513 in which a plurality of receiving antennas 51 is formed, and a main body 428 that houses a shield 54D.

Here, the antenna device 5A includes an antenna cable 52 and a connector portion 53, and the base 513 instead of the above-described plurality of receiving antennas 51. In the base 513, the plurality of antenna patterns 511 to receive a wireless signal from the capsule endoscope device 3, and a transmission pattern (not illustrated) to transmit an electric signal from each antenna pattern 511 to the antenna cable 52 are formed. In the antenna device 5A, an arrangement of the antenna patterns 511 in the base 513 is previously determined. Thus, it is possible to detect a position of the capsule endoscope device 3 on the basis of a wireless signal acquired by itself and the arrangement in the base 513 with respect to the subject 2.

The main body 428 has a saclike shape formed of cloth or the like. The main body 428 houses the base 513 and the shield 54D. The main body 428 has an openable/closable opening formed by utilization of a snap fastener, a slide fastener, or a hook and loop fastener, for example.

The shield 54D has a size with which it is possible to cover, when being housed in the main body 428, all of the antenna patterns 511 formed in the base 513. The shield 54D according to the present fourth embodiment has a size with which it is possible to cover the base 513. When the antenna holder 4A is attached to the subject 2, the shield 54D is arranged on a side opposite to the subject 2 with respect to the base 513. That is, the shield 54D covers all of the antenna patterns 511 by covering a face on an opposite side of the subject 2 side in the base 513.

In the above-described fourth embodiment, the main body 428 houses the base 513 in which the plurality of antenna patterns 511 is formed and the shield 54D, and the shield 54D covers the face on the opposite side of the subject side of the base 513. According to the present fourth embodiment, the antenna patterns 511 formed in the base 513 is shielded and a noise from the outside is reduced, whereby it is possible to control deterioration in communication quality while controlling a necessary amount of a material.

Also, similarly to the above-described first embodiment, according to the above-described present fourth embodiment, it is possible to reduce discomfort of a subject due to sticking of a protective suit, or the like and to control a production and distribution cost compared to a case where a protective suit is used.

### First Modification Example of Fourth Embodiment

FIG. 20 is a partial sectional view schematically illustrating a configuration of a main portion of an antenna holder according to the first modification example of the fourth embodiment of the disclosure. In the above-described fourth embodiment, it has been described that a shield 54D is housed in a main body 428. However, a shield 54D is provided outside a main body 428 in the present first modification example. The antenna holder according to the present first modification example includes the above-described belt portion 41, and an antenna attachment portion 421. The antenna attachment portion 421 includes a main body 428 which houses a receiving antenna 51 and to which a shield 54D is attached on an outer surface that is on an opposite side of a subject side when the antenna holder is attached to a subject 2. The shield 54D is attached to the main body 428 by a publicly known fixing means such as an adhesive or a button.

The main body 428 holds a base 513 in which a plurality of antenna patterns 511 is formed, and the shield 54D is provided on the outer surface of the main body 428 in such a manner as to cover a face on the opposite side of the subject side of the base 513 also in the present first modification example. Thus, each antenna pattern 511 is shielded with the shield 54D, and it is possible to control deterioration in communication quality while controlling a necessary amount of a material.

### Second Modification Example of Fourth Embodiment

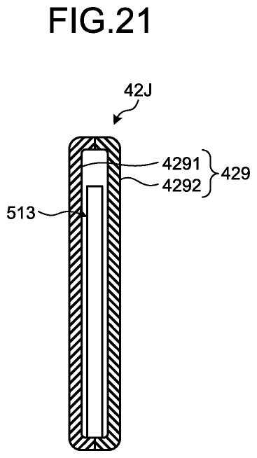

FIG. 21 is a partial sectional view schematically illustrating a configuration of an antenna holder according to the second modification example of the fourth embodiment of the disclosure. In the above-described fourth embodiment, it has been described that a shield 54D is separated from a main body 428. However, a shield is included in a part of a main body in the present second modification example. An antenna attachment portion 42J according to the present second modification example includes a main body 429 to house a base 513.

The main body 429 has a saclike shape. The main body 429 is formed of cloth or the like, and includes a partial main body 4291 included in a part of the saclike shape, and a shielding portion 4292 that has a mesh shape formed of a conductive fiber and that forms the saclike shape with the partial main body 4291. In the main body 429, the partial main body 4291 is arranged on a subject 2 side when the antenna holder is attached to a subject 2. In the present second modification example, the shielding portion 4292 is placed on a face on an opposite side of the subject 2 side of receiving antennas 51, and covers faces on the subject 2 side of all of the receiving antennas 51. The shielding portion 4292 corresponds to a shield.

According to the present second modification example, the main body 429 holds the base 513 in which a plurality of antenna patterns 511 is formed, and the shielding portion 4292 forms an outer surface on the opposite side of the subject side of the main body 429 and covers a face on the opposite side of the subject side of the base 513. Thus, the face on the opposite side of the subject 2 side of the base 513 is shielded, and it is possible to control deterioration in communication quality while controlling a necessary amount of a material.

In the above, embodiments of the disclosure have been described. However, the disclosure should not be limited only to the above-described embodiments and modification examples. The disclosure is not limited to the above-described embodiments and modification examples and may include various embodiments within the scope of a technical idea described in claims. Also, configurations of the embodiments and the modification examples may be arbitrarily combined.

Note that it has been described in the above-described first to fourth embodiments that a shield covers a base in which an antenna pattern is formed. However, as long as a region in which an antenna pattern is formed is covered, an effect can be acquired. Thus, a shield can be applied as long as at least a part corresponding to an antenna pattern-formed region has a configuration with which a shielding effect can be acquired with a conductive fiber or the like.

Also, it has been described in the above-described first to fourth embodiments that a capsule endoscope device 3 is an example of a medical device. However, a medical device is not limited to this. For example, a device that is inserted into a subject, acquires pH information, and outputs the information as a wireless signal may be used.

As described above, an antenna holder and an antenna unit according to the disclosure is useful for controlling deterioration in communication quality while controlling a necessary amount of a material.

According to the disclosure, it is possible to control deterioration in communication quality while controlling a necessary amount of a material.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An antenna holder configured to hold a receiving antenna including at least one receiver to receive a wireless signal transmitted from a medical device inserted into a subject, comprising:
a main body to which the receiving antenna is detachably attached; and
a shield configured to reduce an electromagnetic wave,
wherein when the receiving antenna is attached to the main body, a part of the main body on a subject side, the receiving antenna, and the shield are serially arranged from the subject side.

2. The antenna holder according to claim 1,
wherein the at least one receiver comprises a plurality of receiver,
the receiving antenna includes
the plurality of receivers, and
a plurality of bases in which the plurality of receivers is respectively arranged, and
the shield covers sides opposite to subject sides of all of the plurality of bases.

3. The antenna holder according to claim 2, wherein the shield consists of one member.

4. The antenna holder according to claim 2,
wherein the shield consists of a plurality of members that covers the plurality of bases.

5. The antenna holder according to claim 1,
wherein the receiving antenna includes a base in which the at least one receiver is formed, and
the shield consists of one member and covers a side opposite to a subject side of the base.

6. The antenna holder according to claim 1,
wherein the receiving antenna includes a base in which the at least one receiver is provided, and
the shield has a size with which a face, which is on a side opposite to the subject side in a state of being attached to the subject, among faces of the base is covered.

7. The antenna holder according to claim 1,
wherein the shield is detachably attached to the main body, and
when the receiving antenna and the shield are attached to the main body, the part of the main body on the subject side, the receiving antenna, and the shield are serially arranged from the subject side.

8. The antenna holder according to claim 1,
wherein the shield is arranged on an outer surface of the main body which surface is on a side opposite to the subject side, and
when the receiving antenna and the shield are attached to the main body, the part of the main body on the subject side, the receiving antenna, a part of the main body on the side opposite to the subject side, and the shield are serially arranged from the subject side.

9. The antenna holder according to claim 1,
wherein the shield forms a part of the main body on a side opposite to the subject side, and
when the receiving antenna is attached to the main body, the part of the main body on the subject side, the receiving antenna, and the shield are serially arranged from the subject side.

10. The antenna holder according to claim 1,
wherein the shield detachably holds the receiving antenna, and
when the receiving antenna is attached to the shield, the part of the main body on the subject side, the receiving antenna, and the shield are serially arranged from the subject side.

11. The antenna holder according to claim 1,
wherein the shield is formed of a conductive fiber, an electromagnetic wave shielding material, or a conductive metal.

12. The antenna holder according to claim 1,
wherein at least one processing of corrosion preventing processing and water-resistance processing is performed on the shield.

13. The antenna holder according to claim 1,
wherein the medical device is a capsule endoscope.

14. An antenna unit comprising:
a receiving antenna including at least one receiver to receive a wireless signal transmitted from a medical device inserted into a subject; and
an antenna holder configured to hold the receiving antenna, the antenna holder being attached to the subject,
wherein the antenna holder includes
a main body to which the receiving antenna is detachably attached, and
a shield that reduces an electromagnetic wave, and
when the receiving antenna is attached to the main body, a part of the main body on a subject side, the receiving antenna, and the shield are serially arranged from the subject side.

15. The antenna unit according to claim 14,
wherein the at least one receiver comprises a plurality receiver,
the receiving antenna includes
the plurality of receivers, and
a plurality of bases in which the plurality of receivers is respectively arranged, and
the shield covers sides opposite to subject sides of all of the plurality of bases.

16. The antenna unit according to claim 15,
wherein the shield consists of one member.

17. The antenna unit according to claim 15,
wherein the shield consists of a plurality of members that covers the plurality of bases.

18. The antenna unit according to claim 14,
wherein the receiving antenna includes a base in which the at least one receivers is formed, and
the shield includes one member and covers a side opposite to a subject side of the base.

19. The antenna unit according to claim 14,
wherein the receiving antenna includes a base in which the at least one receiver is provided, and
the shield has a size with which a face, which is on a side opposite to the subject side in a state of being attached to the subject, among faces of the base is covered.
